(19) United States
(12) Patent Application Publication
(10) Pub. No.: US 2017/0059479 A1
(43) Pub. Date: Mar. 2, 2017

(54) Method for Detecting Turbidity Using Coherent Light

(71) Applicant: Parhelion Incorporated, Wilmington, DE (US)

(72) Inventors: Richard REDPATH, Cary, NC (US); James REDPATH, Cary, NC (US)

(21) Appl. No.: 15/245,579

(22) Filed: Aug. 24, 2016

Related U.S. Application Data
(60) Provisional application No. 62/209,096, filed on Aug. 24, 2015.

(51) Int. Cl. G01N 21/31; G01N 33/00

(52) U.S. Cl. CPC G01N 21/31; G01N 33/0027; G01N 2201/06113

(57) ABSTRACT

A method for detecting turbidity using coherent light uses a coherent light emitter such as a laser calibrated to emit a specific wavelength. A light sensor adjacent to the coherent light emitter monitors incoming light to detect the specific wavelength. The coherent light beam will not contact the light sensor unless reflected back to the light sensor, thus detecting the specific wavelength indicates turbidity caused by the presence of smoke, which reflects the coherent light beam back to the sensor. The magnitude of the specific frequency that is detected indicates the amount of smoke detected.

The current application claims a priority to the U.S. Provisional Patent application Ser. No. 62/209,096 filed on Aug. 24, 2015.

### FIELD OF THE INVENTION

The present invention relates to the detection of turbidity using a light source and a sensor. More specifically, the present invention relates to a smoke detection method using coherent light.

### BACKGROUND OF THE INVENTION

Smoke detectors are found in many building as a way for warning occupants of the building as to the presence of a fire. The smoke detectors use sensors to detect the presence of smoke. If sufficient smoke is present, a signal is transmitted.

Using an LED or any source other than coherent light for detection has many issues. The spatial light does not enable precision of measuring turbidity due to its characteristic of illumination as well as its exponential lighting of the smoke called blinding light.

Laser implemented turbidity sensing has traditionally been performed in a closed system. For example, a laser may be located in one part of a room and beam light to a sensor located in another part of the room. This configuration is impractical for use as a smoke detector because of investment in infrastructure setup as well as space and alignment considerations.

The present invention is a method of using lasers in a non-path or unclosed system to detect turbidity, offering the advantage of increased precision as compared to traditional LED detection methods.

### BRIEF DESCRIPTION OF THE DRAWINGS

FIG. 1 is a diagram of a light sensing unit.

FIG. 2 is another diagram of a light sensing unit.

FIG. 3 is a cross section of an optical diffraction grating.

FIG. 4 is a diagram of a light sensing unit illustrating the diffracted light beams being reflected back to the light sensors by smoke.

FIG. 5 is a stepwise flow diagram describing the general steps of the method of the present invention.

FIG. 6 is a stepwise flow diagram describing steps for orientation of the light sensor and for generation of the detection signal.

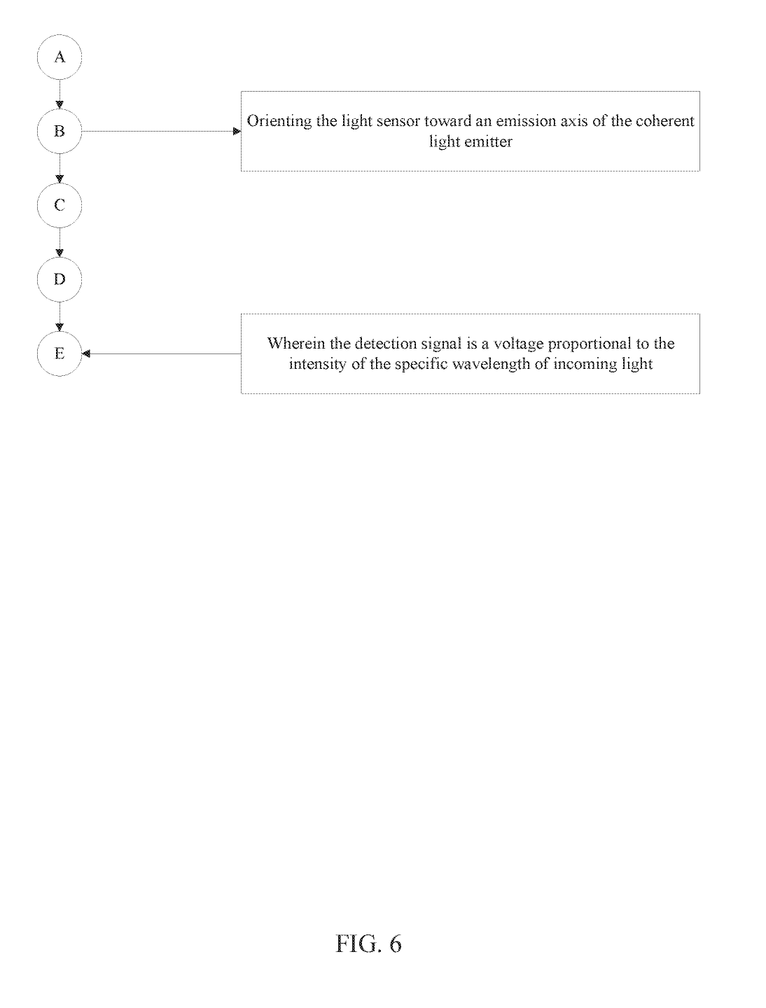

FIG. 7 is a stepwise flow diagram describing steps for utilizing an optical diffraction grating.

FIG. 8 is a stepwise flow diagram describing steps for calibrating the light sensor against ambient light noise.

FIG. 9 is a stepwise flow diagram describing steps for incorporating a processing unit.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

The present invention is a method for detecting turbidity of smoke. In a wireless reporting system, the method of the present invention enables the smoke detector in a room to report to a mobile device which room has smoke and how much. In some embodiments, a wireless communication of smoke detectors for path planning of rescue in open space characteristic for smoke detectors may be used. The main advantage is that the present invention is a non-path, or unclosed system, not requiring interruption of a light signal across a distance to detect turbidity.

Generally, in the present invention, a laser is used to produce multiple coherent laser beams using diffraction mounted at the smoke detector body. A sensor is calibrated for the narrow wavelength of the laser light is mounted adjacent an exit port of laser beams. The sensor and laser light are mounted on the smoke detector. The laser and sensor are positioned in such a way that the sensor will not receive any light from the laser without smoke.

The sensor is calibrated to only sense specific wavelengths. When the sensor receives light of the calibrated wavelength, it will provide a voltage related to the amount of light received. The more light received by the sensor, the greater voltage it will provide.

The laser beams will interact with smoke particles and reflect back to the sensor. The coherent nature of the laser beams will avoid spurious readings as would be for a spatial lighting source such as an LED.

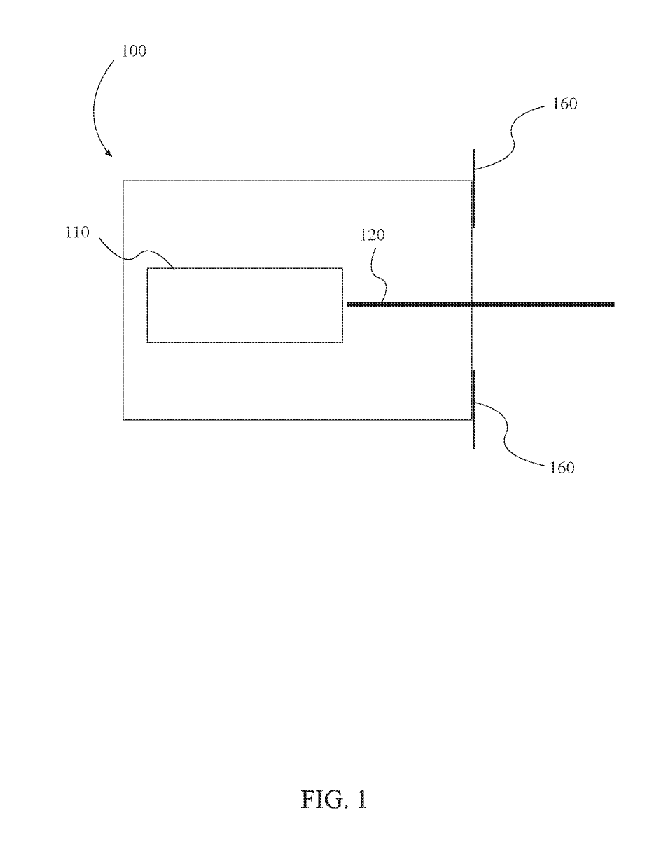

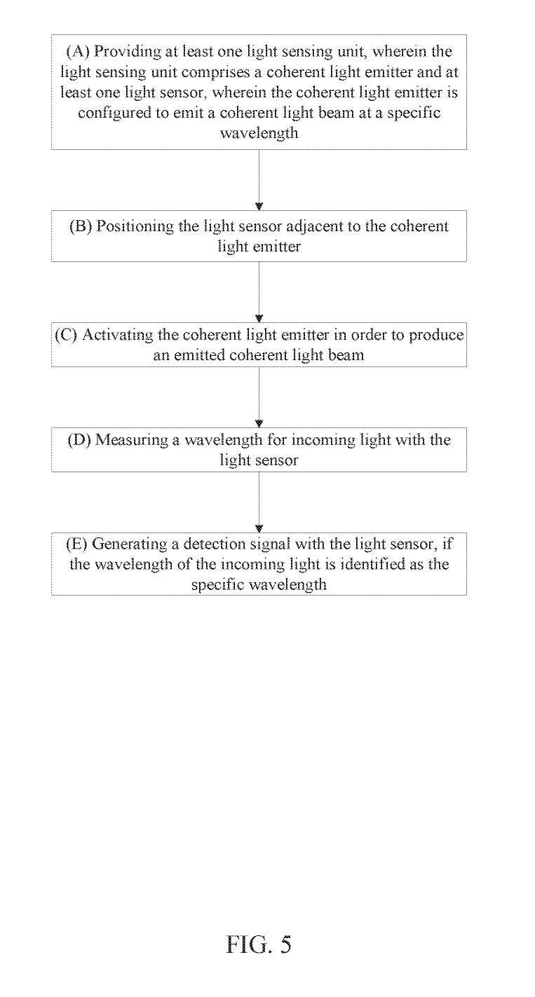

Referring to FIGS. 1 and 5, in the general method of the present invention, at least one light sensing unit 100 is provided. The light sensing unit 100 comprises a coherent light emitter 110 and at least one light sensor 160, wherein the coherent light emitter 110 is configured to emit a coherent light beam 120 at a specific wavelength (Step A of FIG. 5). In the preferred embodiment, the coherent light emitter 110 is a laser diode.

Although it is preferred to use the wavelength of the laser as the criteria for detection, it is further contemplated that frequency of light may alternatively be defined and measured since frequency and wavelength of light are correlated through the equation λν=c, where λ is the wavelength, ν is the frequency and c is the speed of light. Thus, either frequency or wavelength may be measured and its respective counterpart calculated from said equation. Furthermore, any wavelength may be specified as the specific wavelength as desired, from infrared to ultraviolet. In one embodiment, the wavelength is 450 nanometers.

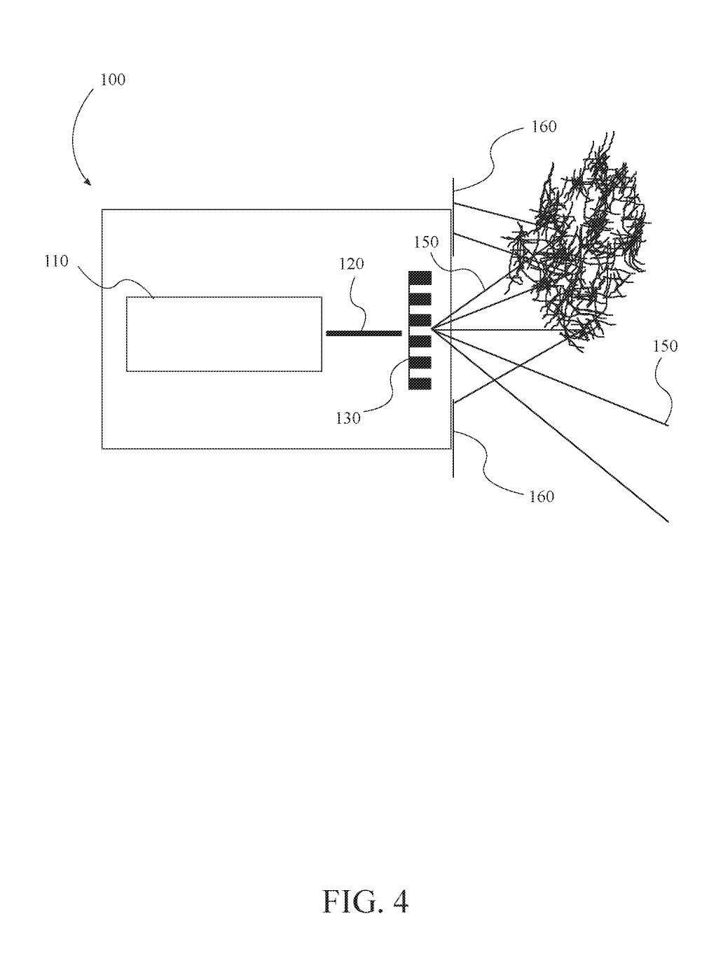

The light sensor 160 is positioned adjacent to the coherent light emitter 110 (Step B of FIG. 5). The light sensor 160 may be positioned around the exit port of the coherent light emitter 110, or to the side of the exit port, or behind the coherent light emitter 110, as shown in FIG. 1. Multiple light sensors 160 may be arranged around the coherent light emitter 110 in any desired configuration. The light sensor 160 cannot detect any of the coherent light produced by the coherent light emitter 110 unless it is incident with turbidity in the air, and reflected back toward the light sensor 160. The main criteria for the light sensor 160 is that the light sensor 160 is oriented generally toward an emission axis of the coherent light emitter 110 in order to be able to detect light beams that have been reflected by smoke, as described in FIG. 4. In one embodiment, the sensor is a TAOS TSL257 optical converter, which comprises a photodiode and a transimpedance amplifier on a single monolithic CMOS integrated circuit.

The coherent light emitter is activated continually in order to produce an emitted coherent light beam 120 (Step C of FIG. 5). A wavelength of incoming reflected light is measured with the light sensor 160 (Step D of FIG. 5). The light sensor 160 continually detects light and measures the wavelength of the light. If the wavelength of the incoming reflected light is identified to be the specific wavelength, corresponding with the light emitted by the coherent light emitter 110, a detection signal is generated with the light sensor 160 (Step E of FIG. 5). In the preferred embodiment of the present invention, the detection signal is a voltage proportional to the intensity of the specific wavelength of incoming light (light to voltage). Thus, in addition to the mere presence of smoke, the present invention provides the capability to quantify the amount of smoke detected (light to voltage). The light sensor for detecting and measuring the intensity of the light is a known prior art and is commercially available.

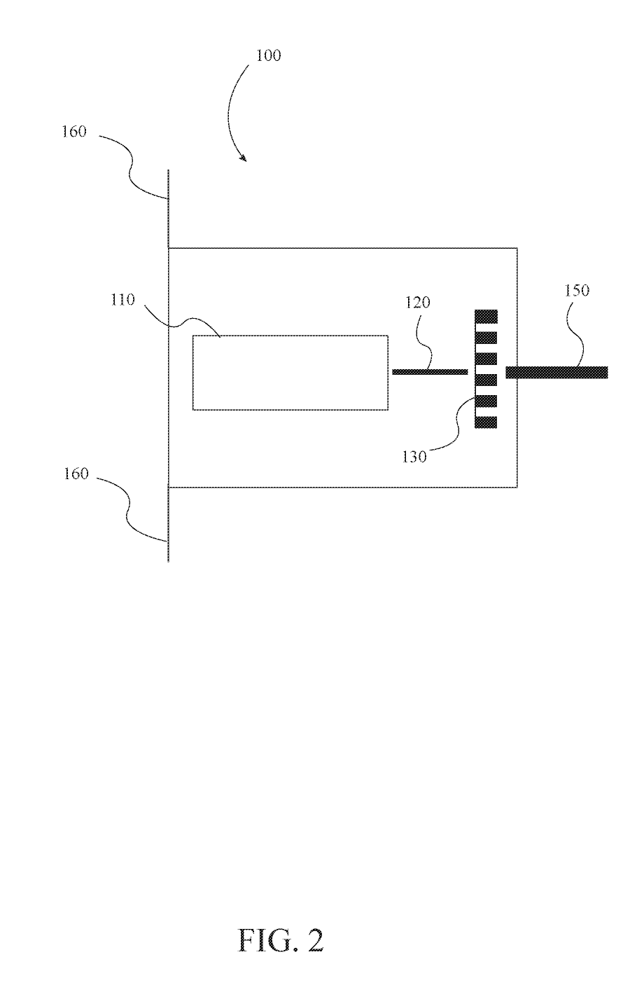

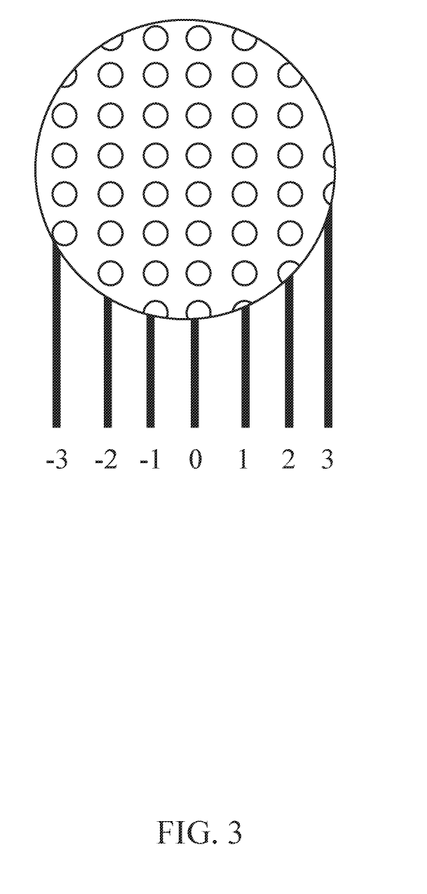

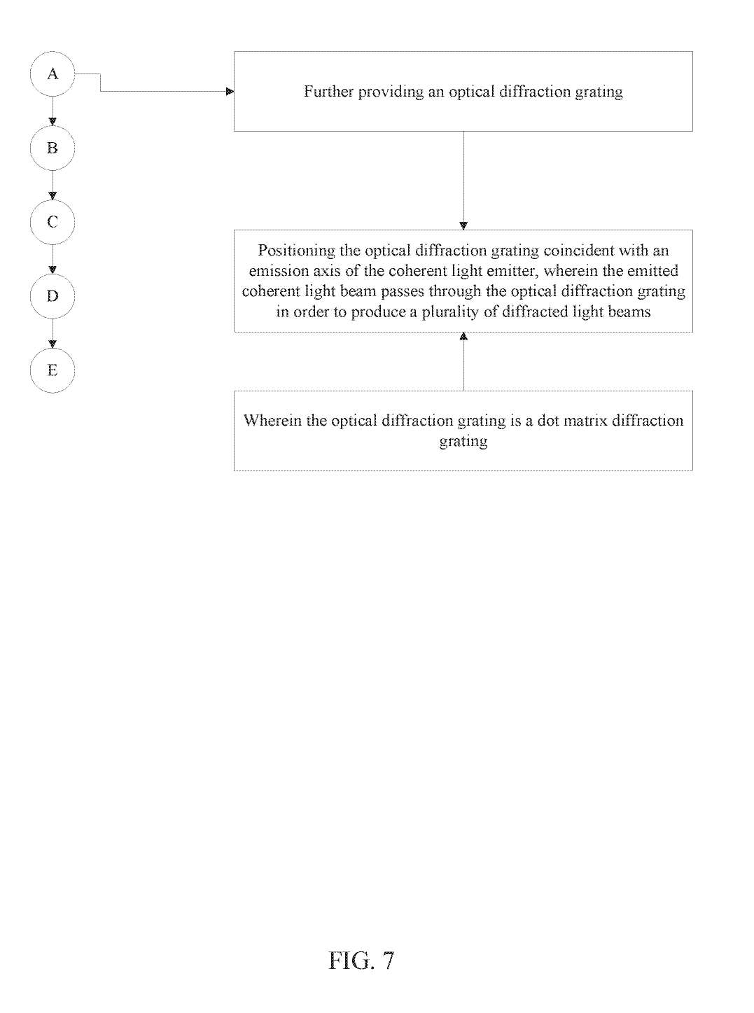

Referring to FIGS. 2-4 and 7, in the preferred embodiment, an optical diffraction grating 130 is further provided. The optical diffraction grating 130 is an optical component with a periodic structure, which splits and diffracts lighting into several beams traveling in different directions. The directions of these beams depend on the spacing of the grating and the wavelength of light so that the grating acts as a dispersive element. The optical diffraction grating 130 is positioned coincident with the emission axis of the coherent light emitter 110, in front of the exit port, so that the emitted coherent light beam 120 passes through the optical diffraction grating 130 in order to produce a plurality of diffracted light beams 150. The optical diffraction grating 130 may comprise any number of designs and patterns. In the preferred embodiment, however, the optical diffraction grating 130 is a dot matrix diffraction grating 130. A cross section of an example optical diffraction grating 130 is shown in FIG. 3. The diffracted light beams 150 should spread out in a conical shape in order to flood the immediate area with coherent light for a higher chance of detecting smoke. The more area covered by the diffracted light beams 150, the greater the chance of detecting smoke when it is present. FIG. 4 shows an illustration of the diffracted light beams 150 being reflected by smoke back to the sensors 160.

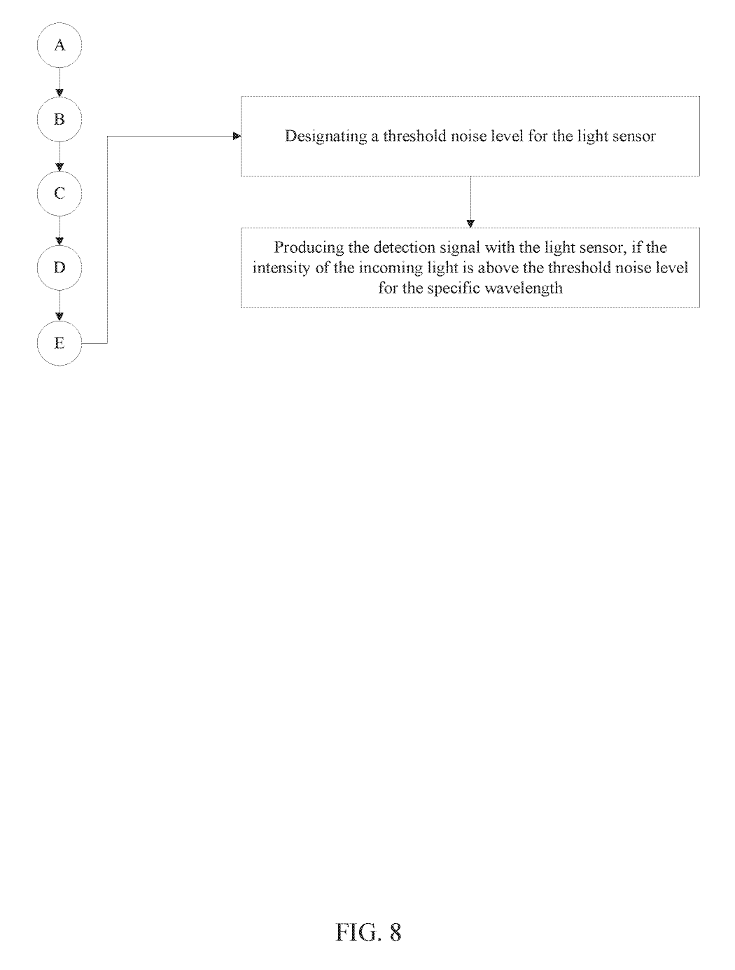

As shown in FIG. 8, after installation in a room, the light sensing unit 100 should be calibrated for any ambient light noise in the room when there is no smoke present. A small amount of light of the specific wavelength may be present, and this should be accounted for to prevent false positives. The amount of light of the specific wavelength under normal, non-smoky conditions is measured by the light sensor 160, and a threshold noise level is designated for the light sensor 160 according to the ambient light level of the specific wavelength. The detection signal is only produced by the light sensor 160 if the intensity of the incoming light detected by the light sensor 160 is above the threshold noise level for the specific wavelength.

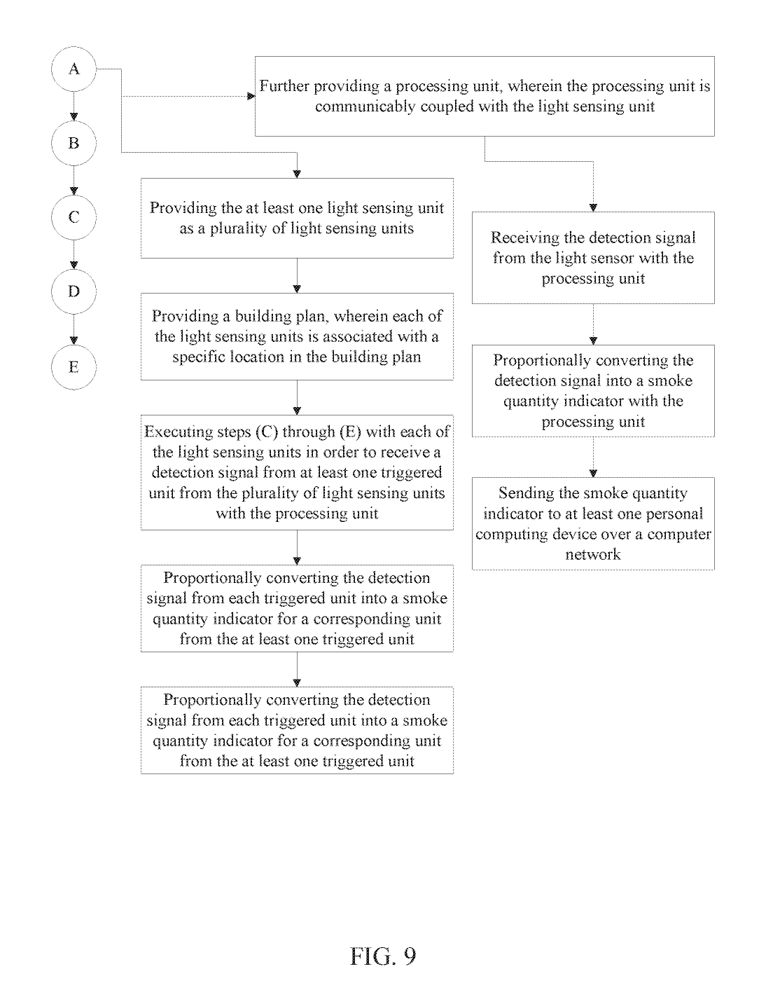

Referring to FIG. 9, in one embodiment, a processing unit is further comprised. The processing unit may be understood to be any circuit, combination of circuits, microprocessor, computing device or other electronic component or combination of components which can facilitate receiving signals from the light sensing unit 100, execute digital commands and processes on said signals, and produce digital or electronic outputs. The processing unit is communicably coupled with the light sensing unit 100. Any detection signal outputted by the light sensor 160 is received with the processing unit. The processing unit may then perform any designated commands based on the data received. For example, in one embodiment the detection signal is proportionally converted into a smoke quantity indicator with the processing unit. The smoke quantity indicator may be numerical, such as a number on a scale between 0 and 100 indicating the amount of smoke detected, or the smoke quantity indicator may be qualitative such as low, medium or high, or the smoke quantity indicator may be any other kind of indicator. The smoke quantity indicator may be send to at least one personal computing device over a computer network. For example, if the system detects smoke in a room in a house with the light sensing unit 100, the owner of the house receives a notification on their mobile smartphone indicating as such.

In one embodiment, the present invention may be utilized to monitor multiple locations in a building in order to provide robust information of where smoke is present in the building. This may be useful for many purposes, such as enabling path planning for rescue or escape in case of a building fire.

Thus, the at least one sensing unit is provided as a plurality of light sensing units 100. A building plan is further provided, and each of the light sensing units 100 is associated with a specific location in the building plan. For example, one light sensing unit 100 may be positioned in each room in the building, or multiple light sensing units 100 may be positioned in large rooms, and light sensing units 100 may be positioned at each exit.

Steps (C) through (E) are executed with each of the light sensing units 100 in order to receive a detection signal from at least one triggered unit from the plurality of light sensing units 100 with the processing unit. The detection signal from each triggered unit is proportionally converted into a smoke quantity indicator for a corresponding unit from the at least one triggered unit, and each of the smoke quantity indicators is displayed at the specific location of the corresponding unit on the building plan on a display device.

It will be appreciated by one skilled in the art that the method of the present invention can be applied to current smoke detectors.

Although the invention has been explained in relation to its preferred embodiment, it is to be understood that many other possible modifications and variations can be made without departing from the spirit and scope of the invention as hereinafter claimed.

What is claimed is:

1. A method for detecting turbidity using coherent light comprises the steps of:
(A) providing at least one light sensing unit, wherein the light sensing unit comprises a coherent light emitter and at least one light sensor, wherein the coherent light emitter is configured to emit a coherent light beam at a specific wavelength;
(B) positioning the light sensor adjacent to the coherent light emitter;
(C) activating the coherent light emitter to produce an emitted coherent light beam with the specific wavelength;
(D) measuring a wavelength for incoming light with the light sensor; and
(E) generating a detection signal with the light sensor, when the wavelength of the incoming light is identified as the specific wavelength.

2. The method for detecting turbidity using coherent light as claimed in claim 1, wherein the detection signal is a voltage proportional to the intensity of the specific wavelength of incoming light.

3. The method for detecting turbidity using coherent light as claimed in claim 1 comprises the step of:
orienting the light sensor toward an emission axis of the coherent light emitter.

4. The method for detecting turbidity using coherent light as claimed in claim 1 comprises the steps of:
further providing an optical diffraction grating; and
positioning the optical diffraction grating coincident with an emission axis of the coherent light emitter, wherein the emitted coherent light beam passes through the optical diffraction grating in order to produce a plurality of diffracted light beams.

5. The method for detecting turbidity using coherent light as claimed in claim 4, wherein the optical diffraction grating is a dot matrix diffraction grating.

6. The method for detecting turbidity using coherent light as claimed in claim 1 comprises the steps of:
designating a threshold noise level for the light sensor; and
producing the detection signal with the light sensor, if the intensity of the incoming light is above the threshold noise level for the specific wavelength.

7. The method for detecting turbidity using coherent light as claimed in claim 1 comprises the steps of:
further providing a processing unit, wherein the processing unit is communicably coupled with the light sensing unit; and
receiving the detection signal from the light sensor with the processing unit.

8. The method for detecting turbidity using coherent light as claimed in claim 7 comprises the steps of:
proportionally converting the detection signal into a smoke quantity indicator with the processing unit; and
sending the smoke quantity indicator to at least one personal computing device over a computer network.

9. The method for detecting turbidity using coherent light as claimed in claim 7 comprises the steps of:
providing the at least one light sensing unit as a plurality of light sensing units;
providing a building plan, wherein each of the light sensing units is associated with a specific location in the building plan;
executing steps (C) through (E) with each of the light sensing units in order to receive a detection signal from at least one triggered unit from the plurality of light sensing units with the processing unit;
proportionally converting the detection signal from each triggered unit into a smoke quantity indicator for a corresponding unit from the at least one triggered unit; and
displaying each of the smoke quantity indicators at the specific location of the corresponding unit on the building plan on a display device.
